(19) United States
(12) Patent Application Publication
(10) Pub. No.: US 2026/0023067 A1
(43) Pub. Date: Jan. 22, 2026

(54) CHAMBER ASSEMBLY FOR EX-VIVO ELECTRORETINOGRAM WITH FILLETED PERFUSION INLET AND OUTLET

(71) Applicants: Matthew Tarchick, Warren, OH (US); Paul Bonezzi, Hinckley, OH (US); Jordan Renna, Hudson, OH (US)

(72) Inventors: Matthew Tarchick, Warren, OH (US); Paul Bonezzi, Hinckley, OH (US); Jordan Renna, Hudson, OH (US)

(21) Appl. No.: 19/116,776

(22) PCT Filed: Sep. 29, 2023
(86) PCT No.: PCT/US2023/034161

§ 371 Date: Mar. 28, 2025

Related U.S. Application Data
(60) Provisional application No. 63/377,615, filed on Sep. 29, 2022.

(51) Int. Cl. G01N 33/483; C12M 1/00; C12M 1/34; G01N 33/50

(52) U.S. Cl. CPC G01N 33/4836; C12M 29/10; C12M 41/46; G01N 33/5082

(57) ABSTRACT

A chamber assembly including a bottom chamber including a sample assembly carried by a bottom body, the sample assembly including a pedestal extending from the body; and a top chamber including a sample chamber within a top body, the sample chamber adapted to generally mate with the pedestal in an in-use position of the chamber assembly; a perfusion assembly with an inlet extending to the sample chamber and an outlet extending away from the sample chamber; a first filleted surface extending from the inlet to the sample chamber; and a second filleted surface extending from the sample chamber to the outlet.

### CROSS REFERENCE TO RELATED APPLICATIONS

This application claims the benefit of U.S. provisional patent application Ser. No. 63/377,615, filed Sep. 29, 2022, which is incorporated by reference herein.

### FIELD OF THE INVENTION

The present disclosure is directed toward a chamber assembly having filleted surfaces, such as for a perfusion inlet and outlet. The present disclosure is further directed toward a chamber assembly for use as an ex-vivo micro-electroretinogram assembly.

### BACKGROUND

The article “Ex-vivo electroretinograms made easy: performing ERGs using 3D printed components” (Bonezzi et al.; J Physiol. 2020 November; 598 (21): 4821-4842) discloses an electroretinogram (ERG) which can be used to analyze rod and cone photoreceptors of the retina. The extracellular activity from populations of rods and cones generate the negative-going a-wave, while ON-bipolar cells generate positive-going b-waves. The article discloses an ERG with an ex vivo configuration, where retinas are isolated and transretinal photovoltages are recorded at high signal-to-noise ratios. The recording configuration is disclosed as providing high signal-to-noise detection of a-waves (300-600 μV) and b-waves (1-3 mV), and being capable of discerning small (1-2 μV) photovoltages from noise. Another conventional ERG chamber is disclosed in the article “Transretinal ERG recordings from mouse retina: rod and cone photoresponses.” (Kolesnikov et al.; J Vis Exp. 2012 Mar. 14; (61):3424. doi: 10.3791/3424).

When perfusing heated, oxygenated, physiological perfusion solution through an ERG chamber to keep tissue alive, bubbles are often degassed in the perfusion tubing during the process of heating. At least certain conventional designs contain sharp edges at the opening of the perfusion line, which results in dead volume where gas bubbles will accumulate. When bubbles dislodge, they can cause the tissue to be dislodged from instrument. Further, these gas bubbles increase the electrical noise in the recording chamber, making it more difficult to detect electrical signals from the tissue.

Moreover, at least certain conventional ERG chambers have been developed for experimentation on mouse retinae, which are roughly 4 mm to 5 mm in diameter. The ability to test smaller samples remains desirable. It also remains desirable to prevent electrical noise in order to obtain a truer signal.

There remains a need in the art for an improved ex-vivo micro-electroretinogram assembly.

### SUMMARY

In one aspect, a chamber assembly includes a bottom chamber including a sample assembly carried by a bottom body, the sample assembly including a pedestal extending from the body; and a top chamber including a sample chamber within a top body, the sample chamber adapted to generally mate with the pedestal in an in-use position of the chamber assembly; a perfusion assembly with an inlet extending to the sample chamber and an outlet extending away from the sample chamber; a first filleted surface extending from the inlet to the sample chamber; and a second filleted surface extending from the sample chamber to the outlet.

In another aspect, a method of testing a sample includes steps of providing the chamber assembly; applying the sample to the pedestal; fastening the bottom chamber with the top chamber in the in-use position; and testing the sample in the in-use position.

### BRIEF DESCRIPTION OF THE DRAWINGS

Advantages of the present disclosure will become better understood with regard to the following description, appended claims, and accompanying drawings wherein:

FIG. 1 is a perspective view of a chamber assembly according to one or more embodiments of the present invention, showing a top chamber separated from a bottom chamber;

FIG. 2 is a top view of the chamber assembly of FIG. 1;

FIG. 3 is a sectional view of the chamber assembly about line F-F of FIG. 2;

FIG. 4 is a close-up view of circle K of FIG. 3;

FIG. 5 is a top view of the bottom chamber of the chamber assembly of FIG. 1;

FIG. 6 is a bottom view of the top chamber of the chamber assembly of FIG. 1;

FIG. 7 is a front view of the bottom chamber of FIG. 5;

FIG. 8 is a side view of the bottom chamber of FIG. 5;

FIG. 9 is a front view of the top chamber of FIG. 6;

FIG. 10 is a side view of the top chamber of FIG. 6;

FIG. 11 is a sectional view of the top chamber about line A-A of FIG. 9;

FIG. 12 is a close-up view of circle E of FIG. 11;

FIG. 13 is a top view of the top chamber of the chamber assembly of FIG. 1;

FIG. 14 is a sectional view of the top chamber about line C-C of FIG. 13;

FIG. 15 is a close-up view of circle G of FIG. 14;

FIG. 16 is a sectional view of the top chamber about line B-B of FIG. 13;

FIG. 17 is a close-up view of circle H of FIG. 16;

FIG. 18 is a sectional view of the bottom chamber about line D-D of FIG. 5;

FIG. 19 is a close-up view of circle J of FIG. 18;

FIG. 20 is a sectional view of the bottom chamber about line E-E of FIG. 5;

FIG. 21 is a close-up view of circle I of FIG. 5;

FIG. 22 is a close-up front view of a bottom electrode port of the bottom chamber of FIG. 5;

FIG. 23 is a close-up top view of the bottom electrode port of FIG. 22;

FIG. 24 is a close-up perspective view of the bottom electrode port of FIG. 22;

FIG. 25 is a close-up top view of a top electrode port of the top chamber of FIG. 6;

FIG. 26 is a close-up front view of the top electrode port of FIG. 25;

FIG. 27 is a close-up perspective view of the top electrode port of FIG. 25; and

FIG. 28 is a graph showing test results obtained via a chamber assembly according to one or more embodiments of the present invention.

### DETAILED DESCRIPTION

One or more embodiments of the present invention are directed toward a chamber assembly. The chamber assembly can be utilized as an ex-vivo micro-electroretinogram assembly which utilizes principles of ex-vivo electroretinography. Electroretinography is a full field electrophysiological recording technique which measures light-evoked voltage transients in the retina and can be used for scientific and diagnostic insight. The ex-vivo aspect allows tissues to be in a controlled environment while also minimizing external sources of noise. As mentioned in the Background, prior to embodiments of the present invention, aspects of certain conventional electroretinogram chambers, and corresponding tests, remain challenging. Advantageously, embodiments of the present invention reduce gas bubbles by providing one or more filleted surfaces. The intersection of a perfusion inlet with a tissue chamber can be a filleted surface, and the intersection of the tissue chamber with a perfusion outlet can be a filleted surface. By utilizing a filleted surface at these intersections, any degassed bubbles (e.g., carbogen) can be redirected into the perfusion outlet and harmlessly passed through to the exit. This reduces or eliminates accumulation of gas bubbles in the recording chamber. As further advantages of embodiments of the present invention, the size of the tissue chamber and the size of the electrode chamber are reduced. This first serves to reduce the amount of perfusion solution which is between the electrodes (i.e., the solution which is present with the sample), which generally decreases voltage junction transients. Moreover, the distance between electrodes can be reduced and smaller samples can be utilized. These and still further advantages are further discussed herein below.

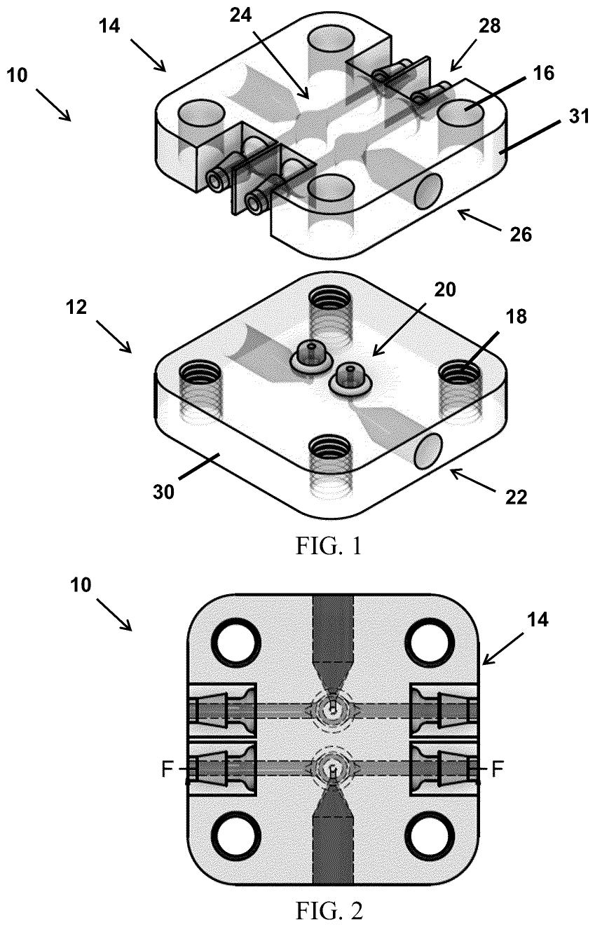

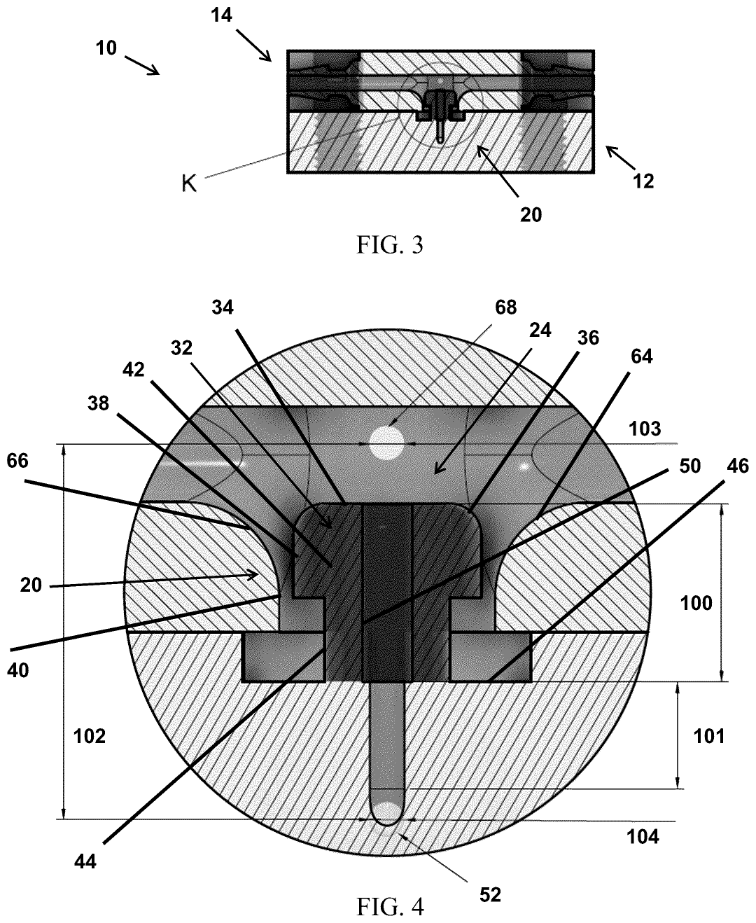

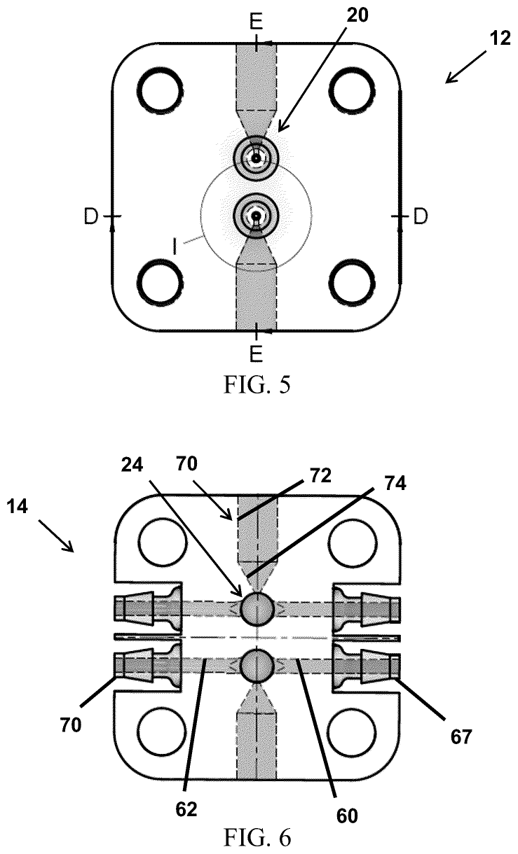

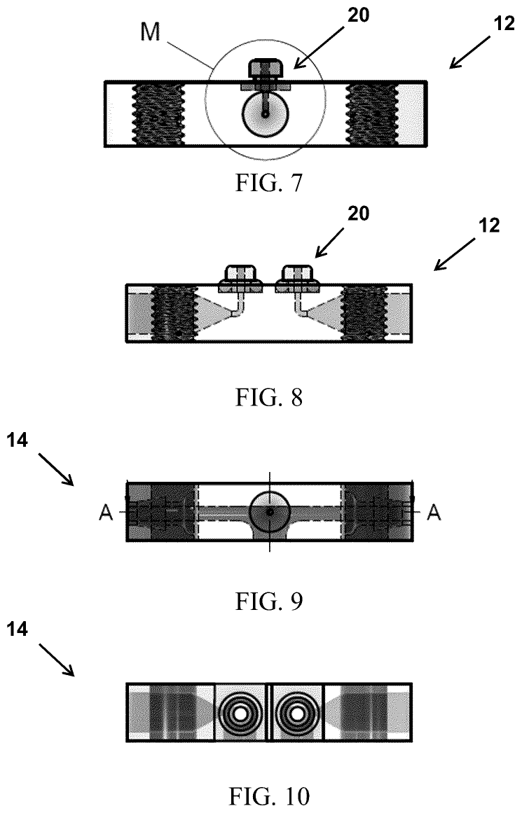

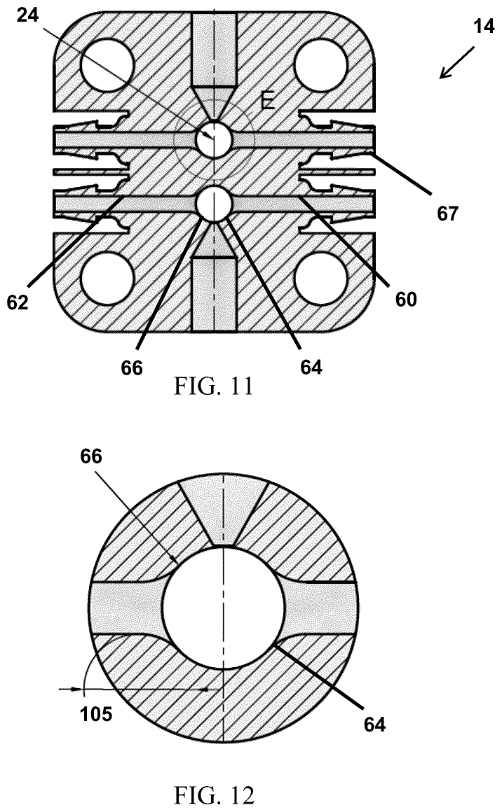

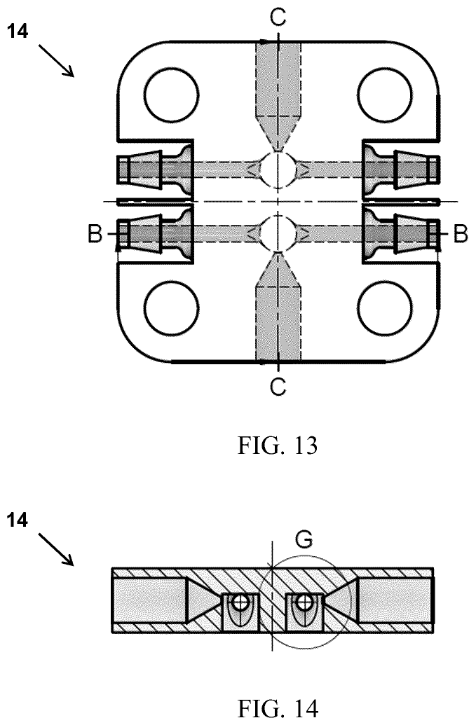

With reference to the Figures, a chamber assembly is generally shown by the numeral 10. Chamber assembly 10, which may also be referred to as ex-vivo micro-electroretinogram assembly 10, ex-vivo electroretinogram 10, electroretinography assembly 10, or assembly 10, includes a bottom chamber 12 and a top chamber 14. FIG. 1 shows bottom chamber 12 separated from top chamber 14, which is the position for loading one or more samples (not shown). In the in-use position, bottom chamber 12 will be secured with top chamber 14 (FIG. 3), such as being fastened together using one or more fasteners, such as screws (not shown). The fasteners can be inserted into unthreaded through holes 16 in top chamber 14 and threaded into threaded through holes 18 of bottom chamber 12. Alternatively, the through holes in top chamber 14 may be threaded and the through holes in bottom chamber 12 may be unthreaded. Also, the threaded holes in either chamber 12, 14 may not be entirely through the chamber 12, 14 as long as sufficient fastening is achieved. While the unthreaded through holes 16 and threaded through holes 18 are generally positioned at the corners of top chamber 14 and bottom chamber 12, other positions may be suitable. Other techniques may be suitable, such as a clamp. Top chamber 14 may also be semi-permanently fixed with bottom chamber 12, such as via a hinge.

Bottom chamber 12, which may also be referred to as bottom component 12, includes one or more sample assemblies 20, which may also be referred to as a pedestal assembly 20, for receiving the one or more samples to be tested. Bottom chamber 12 further includes a bottom electrode assembly 22 coupled with a respective sample assembly 20.

Top chamber 14, which may also be referred to as top component 14, includes one or more sample chambers 24 which generally mate with the respective sample assembly 20 in the in-use position. Top chamber 14 further includes a top electrode assembly 26 coupled with a respective sample chamber 24. Top chamber 14 further includes a perfusion assembly 28 coupled with a respective sample chamber 24.

It should be appreciated that assembly 10 includes a respective set of components. That is, for every one sample assembly 20 which is desired, assembly 10 should generally also include a corresponding one bottom electrode assembly 22, one sample chamber 24, one top electrode assembly 26, and one perfusion assembly 28. While assembly 10 is shown with two sets of respective components, other amounts may be suitable, such as one set of respective components or three sets of respective components. The assembly 10 is not limited to one, two, or three sets, but could utilize any suitable number and arrangement with proper scaling.

Bottom chamber 12 includes a body 30 which includes or carries the components of bottom chamber 12, including the one or more sample assemblies 20. The two sample assemblies 20 are shown in the Figures as being generally centered about body 30, though other arrangements are suitable. Sample assembly 20 includes a pedestal 32 (FIG. 4) where a sample (e.g., retinal tissue) will be placed on a top surface 34 thereof. An edge 36 of the pedestal 32 can be curved, which can allow for a better contour to those samples which are naturally curved. Curved edge 36 can also help to guide a side 38 of pedestal 32 for general mating positioning with sample chamber 24. Side 38 of pedestal 32 may or may not contact an inner portion 40 of the sample chamber 24. In other embodiments, the edge of pedestal 32 may be flat (not shown), such as where testing is desired for a flatter sample.

Pedestal 32 includes an upper wider portion 42 which holds the sample. The upper wider portion 42 should be sized as to receive a sample of a desired size, which can be a relatively smaller size. Exemplary samples include zebrafish retinae, which have a diameter of about 1 mm to about 2 mm, and human retinal organoids, which have a diameter of about 1 mm. Other exemplary samples include retinal tissue, cardiac tissue, brain tissue, other organoids, and stem cells.

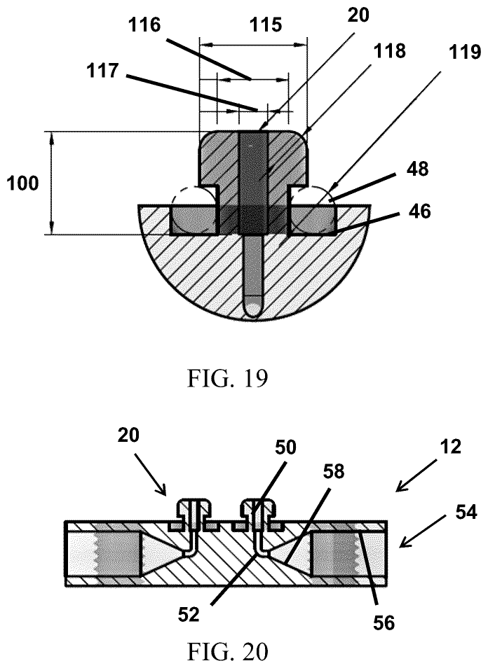

Below wider portion 42 is a narrower portion 44 which is at least partially positioned within an O-ring bore 46, which may be referred to as an O-ring gland 46. O-ring gland 46 receives an O-ring 48 (FIG. 19). When the bottom chamber 12 and top chamber 14 are fastened together, O-ring 48 compresses into the O-ring gland 46 and helps to seal the overall assembly 10 and sample portion thereof from the external environment. An exemplary material for the O-ring 48 is silicon.

Pedestal 32 includes a through hole 50 for allowing an electrode (not shown) to be positioned below the sample by placing the electrode in bottom electrode assembly 22. Through hole 50 can be positioned centrally within pedestal 32. Through hole 50 extends downward to a through hole 52 within bottom chamber 12. The combination of through hole 50 and through hole 52 may be referred to as a bottom electrode aperture. Through hole 52 may have a 90 degree turn before entering a bottom electrode port 54 (FIG. 20). The electrode itself would generally be positioned at the bottom portion of through hole 52 and would not make the 90 degree turn. In other embodiments, where body 30 is thicker, bottom electrode assembly 22 may be positioned below pedestal 32.

Bottom electrode port 54 includes a generally cylindrical portion 56 which extends to a tapered cone portion 58. The tapering of the tapered cone portion 58 generally serves to remove sharp edges and serves as a transition between through hole 52 and electrode port 54. When initially preparing the chamber assembly 10 for testing, an injection of perfusion solution into the electrode port 54 can be utilized to remove excess air therefrom. Without the tapering of the tapered cone portion 58, gas bubbles may form at any sharp edges which would otherwise be present. The tapering generally prevents gas bubbles. The electrode port 54 can be sized to minimize the volume of solution needed to bathe the bottom electrode assembly 22.

For further prevention of gas bubbles, electrode port 54 can be unthreaded as shown in the Figures. Where electrode port 54 is unthreaded, the respective electrode can be wedged in for securing the electrode in position. The small grooves within threading may otherwise accumulate bubbles.

The bottom electrode assembly 22 is shown in the Figures as being generally centered relative to a length of bottom chamber 12, though other arrangements are suitable. The bottom electrode assembly 22 and the corresponding pedestal 32 should be electrically isolated from any other electrode assemblies 22 and pedestals 32 which are present. The bottom electrode assembly 22 and pedestal 32 should also be electrically isolated from the outside environment.

The bottom electrode assembly 22 can be sized to generally reduce the volume thereof. The reduction in volume generally serves to reduce the extra voltage transients that result in electrical noise. For the testing, a higher signal-to-noise is favorable in order to obtain more desirable results. That is, higher noise will generally obscure the desired signal.

Turning back to top chamber 14, top chamber 14 includes a body 31 (FIG. 1) which includes or carries the components of top chamber 14, including the one or more perfusion assemblies 28. Perfusion assemblies 28 are adapted to receive a perfusion solution therethrough during a sample test. Perfusion assembly 28 includes an inlet 60 extending to the sample chamber 24 and an outlet 62 extending away from the sample chamber 24. It should be appreciated that the inlet 60 and outlet 62 are interchangeable depending on the direction of flow of the perfusion fluid. The diameter and/or length of the inlet 60 and outlet 62 can be sized as to reduce the amount of perfusion fluid required, while also preventing gas bubbles from being stuck therein. For example, a smaller diameter will reduce the amount of perfusion fluid required but the diameter should not be so small as to cause gas bubbles.

The transition between the inlet 60 and the sample chamber 24 and the transition between the outlet 62 and the sample chamber 24 include filleted surfaces. The transition between the inlet 60 and the sample chamber 24 includes a first filleted surface 64. Said another way, the first filleted surface 64 generally extends from the inlet 60 to the sample chamber 24. The transition between the outlet 62 and the sample chamber 24 includes a second filleted surface 66. Said another way, the second filleted surface 66 generally extends from the outlet 62 to the sample chamber 24. The first filleted surface 64, which may be referred to as inlet fillet 64, and the second filleted surface 66, which may be referred to as outlet fillet 66, can be symmetrical.

The fillets of inlet fillet 64 and outlet fillet 66 include rounding the respective interior corners and can be described by a radius. Adding the fillets generally serves to provide the perfusion solution as a laminar flow from the inlet 60 into the sample chamber 24. The laminar flow generally serves to reduce dead volume. Bubbles that flow into the sample chamber 24 would otherwise get caught in any dead volume, so the fillets of inlet fillet 64 and outlet fillet 66 serves to prevent bubbles from accumulating. An exemplary shape for the fillets is a truncated ellipse, which would include elongation towards the bottom and truncation at the top. Other shapes, such as generally spherical, can be utilized.

As mentioned above, perfusion assemblies 28 are adapted to receive a perfusion solution therethrough during a sample test. Exemplary perfusion solutions include Ames' medium, Ringer's solution, and Locke's medium. The perfusion solution can include a pharmacological agent as an additive for testing how the sample responds to the pharmacological agent. The perfusion solution can include other additives which may be generally known to the skilled person. The perfusion solution can be bubbled with a gas such as carbogen. An exemplary carbogen includes 95% O₂ and 5% CO₂. Perfusion assembly 28 can include inlet and outlet tube connectors 67 for assistance with suitably securing inlet and outlet tubes or hoses.

As suggested above, the sample chamber 24, which may also be referred to as tissue cavity 24, is positioned between the inlet 60 and outlet 62 of perfusion assembly 28. The sample chamber 24 is adapted to generally mate with the pedestal 32 in an in-use position. As such, the sample will be positioned within the sample chamber 24 in the in-use position for testing.

The sample chamber 24 can be sized to generally reduce the volume thereof. The reduction in volume generally serves to reduce the amount of perfusion fluid which is needed. The reduction in volume also serves to generally reduce the extra voltage transients that result in electrical noise.

In the in-use testing position, sample chamber 24 will receive a top electrode (not shown) by way of a top electrode aperture 68 of top electrode assembly 26. That is, the top electrode will be positioned above the sample. Top electrode aperture 68 and top electrode assembly 26 are generally positioned to the side of sample chamber 24. In other embodiments, where body 31 is thicker, top electrode assembly 26 may be positioned above sample chamber 24.

Top electrode aperture 68 extends to a top electrode port 70 (FIG. 6). Top electrode port 70 includes a generally cylindrical portion 72 which extends to a tapered cone portion 74. The tapering of the tapered cone portion 74 generally serves to remove sharp edges and serves as a transition between top electrode aperture 68 and electrode port 70. The tapering of the tapered cone portion 74 generally serves to prevent gas bubbles from forming at any sharp edges which would otherwise be present. The tapered cone portion 74 is shorter than tapered cone portion 58 because of the space taken up by sample chamber 24.

For further prevention of gas bubbles, top electrode port 70 can be unthreaded as shown in the Figures. Where top electrode port 70 is unthreaded, the respective electrode can be wedged in for securing the electrode in position. The small grooves within threading may otherwise accumulate bubbles.

The top electrode assembly 26 is shown in the Figures as being generally centered relative to a length of top chamber 14, though other arrangements are suitable. The top electrode assembly 26 should be electrically isolated from any other electrode assemblies 26 which are present. The top electrode assembly 26 should also be electrically isolated from the outside environment. The top electrode assembly 26 can be sized to generally reduce the volume thereof. The reduction in volume generally serves to reduce the extra voltage transients that result in electrical noise.

The design of assembly 10 generally serves to reduce the distance between top electrode assembly 26 and bottom electrode assembly 22. Said another way, it is desirable to reduce the distance between the electrodes, which can be reduced down to about 6 mm. Again, reducing this path between electrodes causes fewer extra transients which causes lower noise and therefore results in a higher signal-to-noise ratio.

The material used to make assembly 10 can be any suitable non-conductive material. Exemplary non-conductive materials include plastic and glass. The material used to make assembly 10 can be clear, transparent, or translucent where light is desired to be passed therethrough for a test. For example, testing a retina will include flashing light through assembly 10. Having a clear, transparent, or translucent property will also allow for the observation of whether gas bubbles are present. For testing other samples, such as cardiac tissue and brain tissue, light may not be utilized, such that assembly 10 can be opaque. The assembly 10 can be made by any suitable technique, such as additive manufacturing or molding.

As mentioned above, an exemplary test for assembly 10 is an electroretinography test. Aspects of electroretinography tests and other suitable tests will be generally known to the skilled person. The article “Ex-vivo electroretinograms made easy: performing ERGs using 3D printed components” (Bonezzi et al.; J Physiol. 2020 November; 598 (21):4821-4842) is incorporated by reference herein for aspects related to suitable testing details.

The assembly 10 and corresponding testing equipment are capable of detecting a-waves (300-600 μV) and b-waves (1-3 mV), and is further capable of discerning small (1-2 μV) photovoltages from noise.

As suggested above, the assembly 10 is suitable for use with fully developed samples by placing the fully developed sample in assembly 10 on the day of an experiment. The assembly 10 is also capable of further developing and/or culturing a sample within the enclosed environment. For example, assembly 10 is capable of culturing organoids on the pedestal 32 in the enclosed environment, over days, weeks, and months, which would further allow for continual and periodic testing during the culture process.

Certain dimensions for components of the assembly 10 of one or more embodiments of the present invention are now provided.

With reference to FIG. 4, dimension 100, which may be referred to as a total pedestal height, may be from about 2.5 mm to about 5 mm, in other embodiments, from about 2.85 mm to about 4.35 mm, and in other embodiments, about 2.85 mm. The wider portion of the pedestal may have a height of from about 1.5 mm to about 3 mm, and in other embodiments, about 1.5 mm. Dimension 101, which may be referred to as a through hole length, may be from about 1 mm to about 3 mm, in other embodiments, from about 1.5 mm to about 2 mm, and in other embodiments, about 1.7 mm. Dimension 102, which may be referred to as an electrode path, may be from about 6 mm to about 12 mm, in other embodiments, from about 6 mm to about 9 mm, and in other embodiments, about 6 mm. Dimension 103, which may be referred to as a top electrode port opening, may be about 0.55 mm. Dimension 104, which may be referred to as a bottom electrode port opening, may be about 0.55 mm.

With reference to FIG. 12, dimension 105, which may be referred to as a filleted horizontal radius, may be from about 3 mm to about 3.2 mm, in other embodiments, about 3.12 mm.

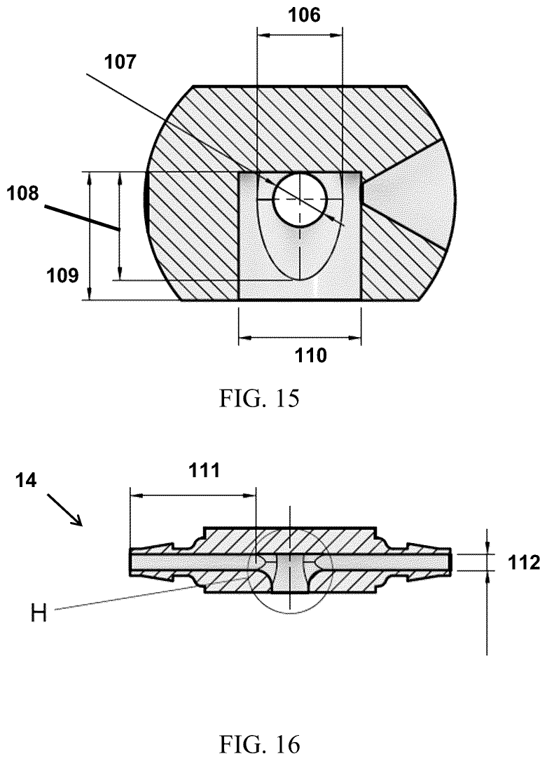

With reference to FIG. 15, dimension 106, which may be referred to as a filleted horizontal length, may be from about 2.3 mm to about 2.4 mm, in other embodiments, about 2.5 mm. Dimension 107 may be from about 1 mm to about 1.5 mm, in other embodiments, about 1.5 mm. Dimension 108, which may be referred to as a filleted vertical length, may be from about 3.02 mm to about 6.02 mm, and in other embodiments, about 3.02 mm. Dimension 109, which may be referred to as a tissue chamber height, may be from about 3.6 mm to about 6.6 mm, and in other embodiments, about 3.6 mm. Dimension 110, which may be referred to as a tissue chamber inner diameter, may be from about 2 mm to about 11 mm, in other embodiments, from about 2.45 mm to about 10.45 mm, and in other embodiments, about 3.45 mm.

With reference to FIG. 16, dimension 111, which may be referred to as a perfusion tube length, may be from about 4 mm to about 13 mm, in other embodiments, from about 4.78 mm to about 11.78 mm, and in other embodiments, about 11.78 mm. Dimension 112, which may be referred to as a perfusion tube diameter, may be from about 0.5 mm to about 2 mm, in other embodiments, from about 0.5 mm to about 1.5 mm, and in other embodiments, about 1.5 mm.

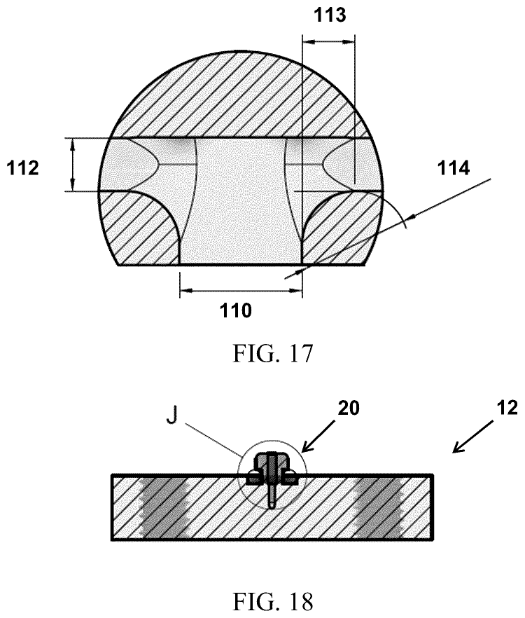

With reference to FIG. 17, dimension 113, which may be referred to as a filleted vertical radius, may be from about 1 mm to about 1.5 mm, and in other embodiments, about 1.5 mm. Dimension 114, may be from about 2 mm to about 4 mm, in other embodiments, from about 2.5 mm to about 3.5 mm, and in other embodiments, about 3 mm.

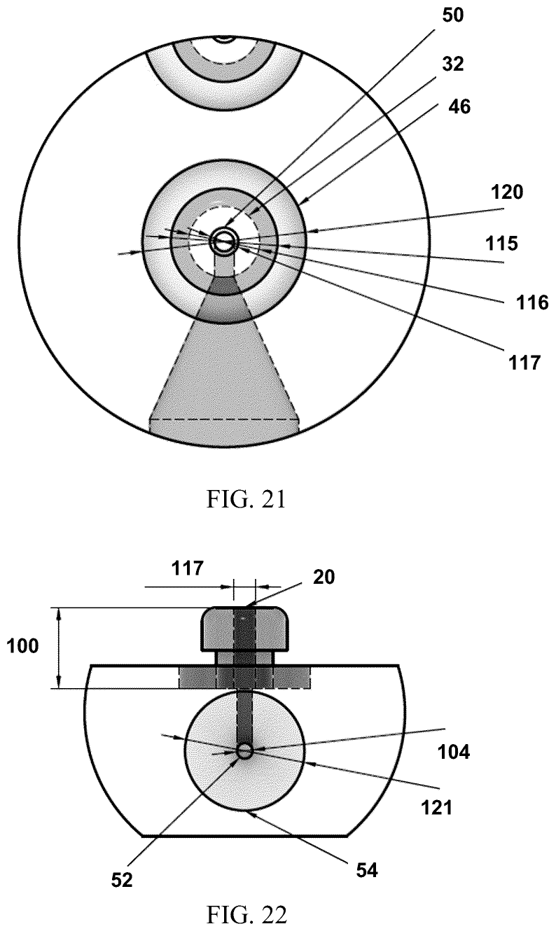

With reference to FIG. 19 and FIG. 21, dimension 115, which may be referred to as a total pedestal width, may be from about 1 mm to about 10 mm, in other embodiments, from about 2 mm to about 9 mm, in other embodiments, from about 1.5 mm to about 3 mm, and in other embodiments, about 3 mm. Dimension 116, which may be referred to as a top pedestal width, may be from about 1 mm to about 10 mm, in other embodiments, from about 1.5 mm to about 9 mm, in other embodiments, from about 1 mm to about 2 mm, and in other embodiments, about 2 mm. Dimension 117, which may be referred to as a bottom electrode aperture, may be from about 0.5 mm to about 0.8 mm, and in other embodiments, about 0.8 mm. Dimension 118, which may be referred to as a pedestal curve or fillet diameter, may be from about 0 mm to about 1.5 mm, in other embodiments, about 1.0 mm, and in other embodiments 0 mm (i.e., flat). Dimension 119, which may be referred to as an O-ring diameter, may be from about 1 mm to about 1.5 mm, and in other embodiments, about 1.3 mm.

With reference to FIG. 21, dimension 120, which may be referred to as an O-ring gland diameter, may be from about 3.1 mm to about 11.6 mm, in other embodiments, from about 3.5 mm to about 5.5 mm, and in other embodiments, about 4.6 mm.

With reference to FIG. 22, dimension 121, which may be referred to as a bottom electrode port inner diameter, may be from about 1 mm to about 5 mm, in other embodiments, from about 2 mm to about 4 mm, and in other embodiments, about 4.2 mm.

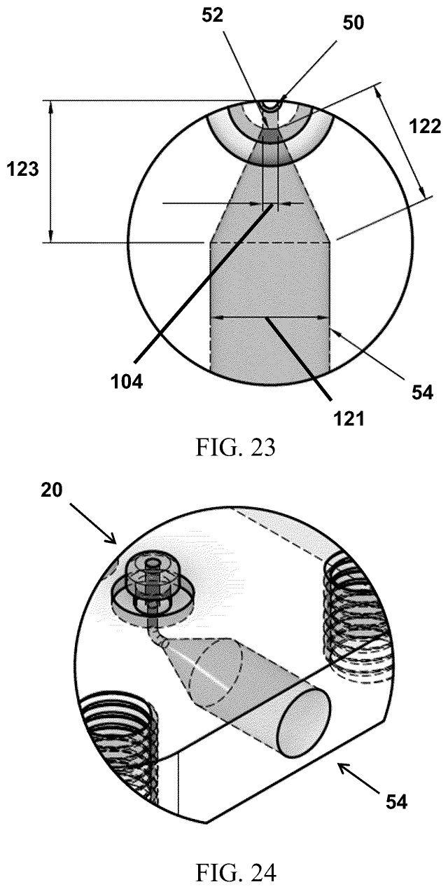

With reference to FIG. 23, dimension 122, which may be referred to as a bottom electrode taper length, may be from about 1.5 mm to about 5 mm, in other embodiments, from about 1.82 mm to about 4.44 mm, and in other embodiments, about 4.4 mm. Dimension 123 may be from about 1 mm to about 6 mm, in other embodiments, from about 2 mm to about 5 mm, and in other embodiments, about 5 mm. An overall length of the bottom electrode port may be from about 2 mm to about 7 mm, in other embodiments, from about 3 mm to about 6 mm, and in other embodiments, about 7 mm.

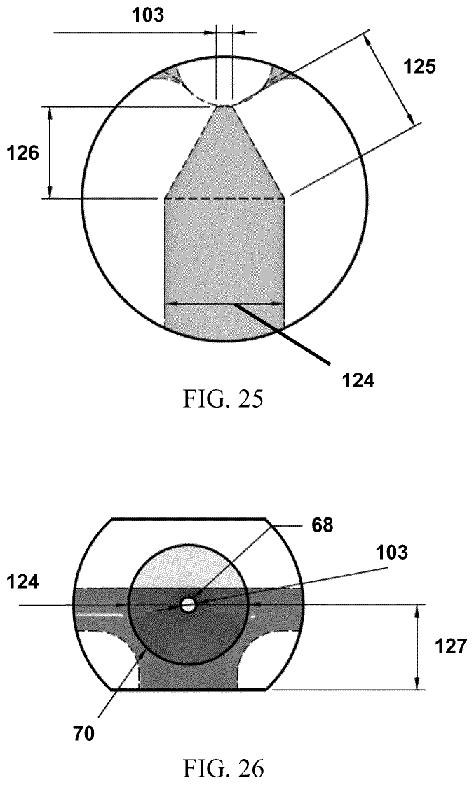

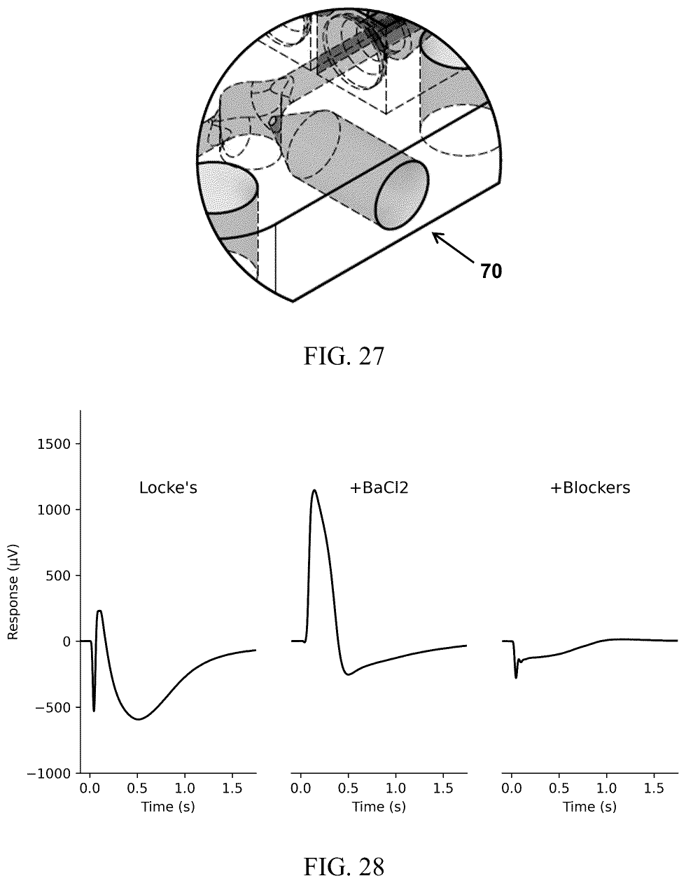

With reference to FIG. 25, dimension 124, which may be referred to as a top electrode port inner diameter, may be from about 1 mm to about 5 mm, in other embodiments, from about 2 mm to about 4 mm, and in other embodiments, about 4.2 mm. Dimension 125, which may be referred to as a top electrode taper length, may be from about 1.5 mm to about 4.5 mm, in other embodiments, from about 1.82 mm to about 3.73 mm, and in other embodiments, about 3.73 mm. Dimension 126 may be from about 1 mm to about 5 mm, in other embodiments, from about 2 mm to about 4 mm, and in other embodiments, about 3.25 mm. An overall length of the top electrode port may be from about 2 mm to about 7 mm, in other embodiments, from about 3 mm to about 6 mm, and in other embodiments, about 7 mm.

With reference to FIG. 26, dimension 127 may be from about 2 mm to about 4 mm, in other embodiments, from about 2.5 mm to about 3.5 mm, and in other embodiments, about 3 mm.

While embodiments of the invention are discussed above, certain exemplary Embodiments are provided here.

Embodiment 1. A chamber assembly including a bottom chamber including a sample assembly carried by a bottom body, the sample assembly including a pedestal extending from the body; and a top chamber including a sample chamber within a top body, the sample chamber adapted to generally mate with the pedestal in an in-use position of the chamber assembly; a perfusion assembly with an inlet extending to the sample chamber and an outlet extending away from the sample chamber; a first filleted surface extending from the inlet to the sample chamber; and a second filleted surface extending from the sample chamber to the outlet.

Embodiment 2. The chamber assembly of Embodiment 1, further comprising a sample placed on the pedestal.

Embodiment 3. The chamber assembly of any of the above Embodiments, where the sample is a retinal tissue, where the chamber assembly is clear, transparent, or translucent.

Embodiment 4. The chamber assembly of any of the above Embodiments, where the sample is a cardiac tissue, brain tissue, organoid, or stem cell.

Embodiment 5. The chamber assembly of Embodiment 4, where the organoid is an undeveloped organoid.

Embodiment 6. The chamber assembly of any of the above Embodiments, where the chamber assembly is coupled with test equipment, where the test equipment is for an ex-vivo electroretinogram test.

Embodiment 7. The chamber assembly of any of the above Embodiments, where the first filleted surface and the second filleted surface are shaped as truncated ellipses.

Embodiment 8. The chamber assembly of any of the above Embodiments, where the sample has a diameter of about 1 mm to about 2 mm.

Embodiment 9. The chamber assembly of any of the above Embodiments, where the pedestal includes an upper wider portion for holding a sample, and a narrower portion below the upper wider portion, where the narrower portion is at least partially positioned within an O-ring bore within the bottom chamber, where the O-ring bore includes an O-ring for sealing the chamber assembly in the in-use position.

Embodiment 10. The chamber assembly of Embodiment 9, where the upper wider portion includes a curved edge.

Embodiment 11. The chamber assembly of any of the above Embodiments, further comprising an upper electrode assembly and a lower electrode assembly, the upper electrode assembly including a first generally cylindrical portion extending to a first tapered cone portion, the lower electrode assembly including a second generally cylindrical portion extending to a second tapered cone portion.

Embodiment 12. The chamber assembly of Embodiment 11, where the first tapered cone portion is shorter in length than the second tapered cone portion.

Embodiment 13. The chamber assembly of Embodiment 11 or 12, the upper electrode assembly including a top electrode port opening having a diameter of about 0.55 mm, the lower electrode assembly including a bottom electrode port opening having a diameter of about 0.55 mm.

Embodiment 14. The chamber assembly of any of Embodiments 11 to 13, the upper electrode assembly including an overall top port diameter of from about 1 mm to about 5 mm, the lower electrode assembly including an overall bottom port diameter of from about 1 mm to about 5 mm.

Embodiment 15. The chamber assembly of any of Embodiments 11 to 14, the upper electrode assembly and the lower electrode assembly defining an electrode path with a length of from about 6 mm to about 12 mm.

Embodiment 16. The chamber assembly of any of the above Embodiments, the pedestal having a top portion which has a width of from about 1 mm to about 2 mm in order to receive a sample having a diameter of from about 1 mm to about 2 mm.

Embodiment 17. The chamber assembly of any of the above Embodiments, the pedestal having an overall height of from about 2.5 mm to about 5 mm.

Embodiment 18. The chamber assembly of any of the above Embodiments, the sample chamber having a height of from about 3.6 mm to about 6.6 mm.

Embodiment 19. The chamber assembly of any of the above Embodiments, the sample chamber having an inner diameter of from about 2 mm to about 11 mm.

Embodiment 20. A method of testing a sample, the method comprising steps of providing the chamber assembly of any of the above Embodiments; applying the sample to the pedestal; fastening the bottom chamber with the top chamber in the in-use position; and testing the sample in the in-use position.

Embodiment 21. The method of Embodiment 20, where the sample is a retinal tissue, where the step of testing is an ex-vivo electroretinogram test.

Embodiment 22. The method of Embodiment 20, where the sample is an undeveloped organoid, the method further comprising a step of further developing the undeveloped organoid while the chamber assembly is in the in-use position.

Embodiment 23. The method of Embodiment 22, where the step of further developing the undeveloped organoid occurs for at least one week or at least month.

Embodiment 24. The method of Embodiment 22 or 23, further comprising a step of further testing the undeveloped organoid during the step of further developing the undeveloped organoid.

Embodiment 25. The method of any of Embodiments 22 to 24, where the step of further developing the undeveloped organoid occurs until the undeveloped organoid is fully developed.

In light of the foregoing, it should be appreciated that the present invention advances the art by providing an improved ex-vivo micro-electroretinogram assembly. While particular aspects of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

An ex-vivo micro-electroretinogram assembly according to one or more embodiments of the present invention was utilized to measure flash responses from electroretinography tests for a variety of fluids. The results are shown in FIG. 28.

The first graph (“Locke's”) of FIG. 28 shows a flash response for a Locke's saline solution. The second graph (“+BaCl2”) of FIG. 28 shows the same flash response for the Locke's saline solution having an addition of BaCl₂. The third graph (“+Blockers”) of FIG. 28 shows the same flash response for the Locke's saline solution having an addition of BaCl₂, a glutamate blocker (L-AP4 (L-2-amino-4-phosphonobutyric acid)), and aspartic acid.

Various modifications and alterations that do not depart from the scope and spirit of this invention will become apparent to those skilled in the art. This invention is not to be duly limited to the illustrative examples set forth herein.

1. A chamber assembly comprising
a bottom chamber including a sample assembly carried by a bottom body, the sample assembly including a pedestal extending from the body; and
a top chamber including
a sample chamber within a top body, the sample chamber adapted to generally mate with the pedestal in an in-use position of the chamber assembly;
a perfusion assembly with an inlet extending to the sample chamber and an outlet extending away from the sample chamber;
a first filleted surface extending from the inlet to the sample chamber; and
a second filleted surface extending from the sample chamber to the outlet.

2. The chamber assembly of claim 1, further comprising a sample placed on the pedestal.

3. The chamber assembly of claim 2, where the sample is a retinal tissue, where the chamber assembly is clear, transparent, or translucent.

4. The chamber assembly of claim 2, where the sample is a cardiac tissue, brain tissue, organoid, or stem cell.

5. The chamber assembly of claim 4, where the organoid is an undeveloped organoid.

6. The chamber assembly of claim 1, where the chamber assembly is coupled with test equipment, where the test equipment is for an ex-vivo electroretinogram test.

7. The chamber assembly of claim 1, where the first filleted surface and the second filleted surface are shaped as truncated ellipses.

8. The chamber assembly of claim 2, where the sample has a diameter of about 1 mm to about 2 mm.

9. The chamber assembly of claim 1, where the pedestal includes an upper wider portion for holding a sample, and a narrower portion below the upper wider portion, where the narrower portion is at least partially positioned within an O-ring bore within the bottom chamber, where the O-ring bore includes an O-ring for sealing the chamber assembly in the in-use position.

10. The chamber assembly of claim 9, where the upper wider portion includes a curved edge.

11. The chamber assembly of claim 1, further comprising an upper electrode assembly and a lower electrode assembly, the upper electrode assembly including a first generally cylindrical portion extending to a first tapered cone portion, the lower electrode assembly including a second generally cylindrical portion extending to a second tapered cone portion.

12. The chamber assembly of claim 11, where the first tapered cone portion is shorter in length than the second tapered cone portion.

13. The chamber assembly of claim 12, the upper electrode assembly including a top electrode port opening having a diameter of about 0.55 mm, the upper electrode assembly including an overall top port diameter of from about 1 mm to about 5 mm, the lower electrode assembly including a bottom electrode port opening having a diameter of about 0.55 mm, the lower electrode assembly including an overall bottom port diameter of from about 1 mm to about 5 mm, and the upper electrode assembly and the lower electrode assembly defining an electrode path with a length of from about 6 mm to about 12 mm.

14. (canceled)

15. (canceled)

16. The chamber assembly of claim 1, the pedestal having a top portion which has a width of from about 1 mm to about 2 mm in order to receive a sample having a diameter of from about 1 mm to about 2 mm, the pedestal having an overall height of from about 2.5 mm to about 5 mm, the sample chamber having a height of from about 3.6 mm to about 6.6 mm, and the sample chamber having an inner diameter of from about 2 mm to about 11 mm.

17. (canceled)

18. (canceled)

19. (canceled)

20. A method of testing a sample with the chamber assembly of claim 1, the method comprising steps of
providing the chamber assembly of claim 1;
applying the sample to the pedestal;
fastening the bottom chamber with the top chamber in the in-use position; and
testing the sample in the in-use position.

21. The method of claim 20, where the sample is a retinal tissue, where the step of testing is an ex-vivo electroretinogram test.

22. The method of claim 20, where the sample is an undeveloped organoid, the method further comprising a step of further developing the undeveloped organoid while the chamber assembly is in the in-use position.

23. The method of claim 22, where the step of further developing the undeveloped organoid occurs for at least one week-or at least month.

24. The method of claim 22, further comprising a step of further testing the undeveloped organoid during the step of further developing the undeveloped organoid.

25. The method of claim 22, where the step of further developing the undeveloped organoid occurs until the undeveloped organoid is fully developed.
